(19) United States
(12) Patent Application Publication
(10) Pub. No.: US 2010/0122355 A1
(43) Pub. Date: May 13, 2010

(54) Transgenic Reporter Mouse and Method for Use

(76) Inventors: Neal Paragas, New York, NY (US); Jonathan Matthew Barasch, New York City, NY (US); Andong Qiu, Bronx, NY (US)

Correspondence Address: Hasse & Nesbitt LLC, 8837 Chapel Square Drive, Suite C, CINCINNATI, OH, 45249, US

(21) Appl. No.: 12/504,658

(22) Filed: Jul. 16, 2009

Related U.S. Application Data
(60) Provisional application No. 61/081,167, filed on Jul. 16, 2008.

(51) Int. Cl. A61K 49/00; A01K 67/027; C12N 5/00

(52) U.S. Cl. 800/3; 800/18; 435/325

(57) ABSTRACT

A transgenic mammal, including a transgenic mouse, whose genome comprises a transgene, said transgene comprises a neutrophil gelatinase-associated lipocalin (NGAL) promoter gene operably linked to at least one sequence encoding at least one of a fluorescent or bioluminescent protein, wherein the NGAL promoter gene expression in the mouse can be assayed by bioluminescence or fluorescence imaging.

### GOVERNMENT INTERESTS

None

### FIELD OF THE INVENTION

The present invention relates to noninvasive methods and compositions for detecting, localizing and tracking light-emitting entities and biological events in a mammalian subject.

### BACKGROUND OF THE INVENTION

NGAL (neutrophil gelatinase-associated lipocalin) is a protein that is expressed in massive quantities by the renal tubule when a patient or an animal suffers sepsis and acute kidney injury (Mori et al., 2005, Barasch and Mori, 2004, Mishra et al., 2004, Mishra et al., 2003). Because NGAL is expressed many hours and even many days before serum creatinine rises (Mishra et al., 2005, Wagener et al., 2006), it has been proposed that NGAL is a rapid diagnostic biomarker of sepsis and renal failure. Past findings come from adults (Nickolas et al., 2008) and children (Bennett et al., 2008) and preliminary data shows that neonates also synthesize NGAL.

Currently, the diagnosis of sepsis and renal failure is a retrospective diagnosis because of the non-steady state kinetics of serum creatinine. Large declines in glomerular filtration rate (GFR) may manifest only as a small change in serum creatinine, particularly in the initial 48 hours following acute kidney injury (AKI) (Lameire and Hoste, 2004), and low muscle mass, poor nutritional status, certain medications and co-morbid disease may further suppress the rise in serum creatinine and confound the diagnosis. Highlighting the insensitivity of serum creatinine is the finding that even ‘subclinical’ changes (i.e., elevations of serum creatinine that do not meet established criteria for AKI and hence may be overlooked) (Bellomo et al., 2004) are known to be associated with excess mortality, prolonged hospitalization, readmission and functional decline, and elevated financial costs (Chertow et al., 2005, Lassnigg et al., 2004, Gottlieb et al., 2002, Smith et al., 2003). In addition, not only is serum creatinine insensitive, but when it does become elevated, serum creatinine cannot distinguish between prerenal azotemia, acute kidney injury (AKI) or chronic kidney disease (CKD), because in each of these states serum creatinine may be elevated to the same extent despite the fact that these states connote very distinct conditions. For example, AKI requires hospitalization, but the other two states may not.

Limitations in the serum creatinine provide the rationale for the discovery of kidney proteins that are expressed at the onset of AKI and are more sensitive and specific for the diagnosis of AKI than currently available tests. Preliminary data in adults, children, and neonates show that NGAL will alert the physician to impending sepsis and AKI hours to days before an official diagnosis can be achieved. Additionally, Chronic Kidney Disease (CKD) patients with rapidly advancing kidney failure express urine NGAL, but CKD patients with non-progressive disease have much lower NGAL levels, (Unpublished) indicating that the expression of urine NGAL is stimulated by ongoing, but not by fixed, changes in the kidney (Mori and Nakao, 2007). NGAL concentration does not increase in mice or humans with volume depletion or diuretic therapy (Nickolas et al., 2008), again indicating specificity for tubular damage. These intriguing observations suggest that urine NGAL not only detects AKI, but that it may distinguish types of kidney diseases by its expression level.

Known NGAL biomarker properties include the following: that the amount of NGAL in the blood or urine is proportional to disease intensity, NGAL expression reverses when the disease abates (Mishra et al., 2003) or if the disease is treated (such as antibiotic therapy for sepsis), NGAL exhibits sensitivity and specificity for AKI and “unstable CKD”, and NGAL kinetics of expression and regression are consistent with rapid diagnosis of new onset renal failure. NGAL is a carrier protein with binds organic molecules called siderophores (iron binding cofactors). NGAL chelates iron using a novel cofactor, catechol. It delivers iron to proximal tubule, about 10 ug at steady state, but up to 1 mg during disease. NGAL is a siderophore decoy that has an antimicrobial activity. NGAL chelates iron and arrests bacterial infection by sequestering catecholate-siderophore bound iron. In sum NGAL is a critical component of epithelia against invasive bacteria and may participate in iron scavenging.

The role of NGAL in sepsis and renal failure is now clear. Like most lipocalins, NGAL binds one or more ligands. When the protein is cloned, a reddish hue is noted that is identifiable as a bacterial substance, a siderophore:iron complex called enterochelin that NGAL binds with during cloning (Goetz et al., 2002). While this identification was highly unexpected, the association of NGAL and siderophore is confirmed by assays in vivo, including excess growth of bacteria in NGAL−/− knockout mice (Flo et al., 2004) and the demonstration that the NGAL:enterochelin:Fe complex forms in vivo (Mori et al, 2005). In addition, endogenous urinary siderophores are found that bind NGAL. From these data it is speculated that NGAL plays a critical role in the urinary tract by depriving essential iron from bacteria, and that NGAL is particularly expressed for this purpose when kidney epithelia sense infection and/or tissue destruction. Moreover, the capture of NGAL:siderophore:Fe may recycle iron to viable cells, and might induce growth and repair.

The expression of NGAL has been detected in the nephron by in situ hybridization on frozen sections and was detected in the urine and blood by use of ELISA/Western Blots. In normal mice and humans, NGAL is expressed at low levels by the collecting ducts of the kidney. Upon ischemia-reperfusion injury in mice, NGAL is massively expressed by thick ascending limbs and collecting duct segments of the nephron (Schmidt-Ott et al. 2007, Mori et al 2005) whereas with polymicrobial sepsis (cecal ligation and puncture) NGAL was induced in the proximal tubule, and with obstructive uropathy NGAL was induced predominantly in the medullary collecting ducts. The fact that many parts of the nephron can synthesize NGAL after different stressors is intriguing from the point of view of signal transduction, but additionally this finding is of great interest because NGAL can rapidly detect renal injury of many different types.

NGAL also traffics in the serum and is found in abundance in the urine. During stressful events, but not at baseline, NGAL's rapid expression has indicated its use as a biomarker that over come problems of using creatinine which is delayed in its presentation, and is a superior renal marker than cystatin C. NGAL is also a gene that is expressed in neutrophils and epithelia, including those in the skin, liver, and kidney.

Current techniques for measuring NGAL gene expression in the experimental setting are limited by the requirement to sacrifice the mouse or have laborious collections of urine or blood. This limits the ability to determine expression time course, additive effects of different stressors, or the response to medications or therapies that may be tried in an attempt to terminate renal disease. Assay of mouse urine or blood by standard methods is infeasible for high throughput screening programs. Additionally, because NGAL may be expressed and secreted from different types of cells, sacrifice of the test animal and extensive evaluation is required to determine the source of NGAL induced by a disease, toxin, medication or other stressor. Sacrifice and extensive pathological investigation is unfeasible in high throughput screening and hence the target tissues that express NGAL in a series of mice can not be readily determined. These issues are easily surmounted by creation of a NGAL reporter mouse.

### SUMMARY OF THE INVENTION

The present invention relates to a transgenic mammal, including a transgenic mouse, whose genome comprises a transgene, said transgene comprises a neutrophil gelatinase-associated lipocalin (NGAL) promoter gene operably linked to at least one sequence encoding at least one of a fluorescent or bioluminescent protein, wherein the NGAL promoter gene expression in the mouse can be assayed by bioluminescence or fluorescence imaging.

Fluorescent or bioluminescent protein includes, but is not limited to, infrared-fluorescent proteins (IFPs), mRFP1, mCherry, mOrange, DsRed, tdTomato, mKO, TagRFP, EGFP, mEGFP, mOrange2, maple, TagRFP-T, Firefly Luciferase, Renilla Luciferase and Click Beetle Luciferase.

The protein can include a luciferase protein, including firefly luciferase (Luc2) protein, and a red fluorescent protein, including monomeric red fluorescent protein (mCherry), and the sequence encoding the luciferase protein and the sequence encoding the red fluorescent protein are separated by a spacer, although the proteins can be combined in various ways, and in various combinations to achieve effective emission of light (typically 544 nm-714 mm) from NGAL-reporter promoter activity.

The present invention also provides a progeny of the transgenic mammal, wherein the genomes of said progeny comprise said transgene, and NGAL gene expression in the progeny can be assayed by bioluminescence or fluorescence imaging.

The present invention also provides a method of screening for candidate agent that would cause renal injury, comprising the steps of: (a) contacting the transgenic mammal with an agent; and (b) examining NGAL expression in the transgenic mouse by bioluminescence or fluorescence imaging, wherein increased bioluminescence or fluorescence after treatment with the agent indicates the agent would cause organ or tissue injury or damage.

The present invention also provides a method of screening for candidate agent that would prevent or treat injury to an organ or tissue that is detected by expression of NGAL, comprising the steps of: (a) inducing renal injury in the transgenic mammal; (b) contacting the transgenic mammal with an agent; and (c) examining NGAL expression in the transgenic mammal by bioluminescence or fluorescence imaging, wherein decreased bioluminescence or fluorescence after treatment with the agent indicates the agent would prevent or treat renal injury. The candidate agent can include an antibiotic for infections, a topical steroid for psoriasis, an inhaled steroid and a beta-agonists for lung disease, and a sunscreen for UV light exposure.

The renal injury is renal failure or sepsis.

The present invention also provides a transgenic cell line comprising an neutrophil gelatinase-associated lipocalin (NGAL) promoter gene operably linked to at least one sequence encoding at least one of a fluorescent or bioluminescent protein, wherein the NGAL promoter gene expression in the cell line can be assayed by bioluminescence or fluorescence imaging. The cell line can be a specific type of cell representing a component of an organ or cancer cell, and permits high through-put screening of drugs, mediations, toxins, industrial chemicals, food additives, bacteria or their products, and UV light.

The present invention also relates to a transgenic mammal that also includes a second genetic model, including or such as of genetic models of the kidney (e.g. polycystic kidney, HIV associated nephropathy), the lung (cystic fibrosis, bronchitis), the liver (cirrhosis of different types), sepsis, vasculitis, atherosclerosis, fibrosis whose onset and/or amelioration by therapy can be followed and titrated by the reporter mouse.

The present invention also relates to testing of the effects of extracorporal circuits, including and such as cardiothoracic bypass, dialysis, indwelling catheters in the artery, vein, heart, bladder, rectum, colon, small intestine, stomach, esophagus, tissue spaces such as trachea, peritoneum all of which may induce tissue damage made visible by the NGAL promoter-reporter gene.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, Panels A and B and 1B, Panels C and D shows the generation of a NGAL-diffusion reporter murine model. FIG. 1A, Panel A shows that the luciferase-mCherry di-fusion reporter gene together with a Loxp-Neomycin-Loxp cassette (LNL) was knocked into a site right before the translation initiation codon (ATG) of the NGAL gene using BAC recombineering. Subsequent BAC recombineering introduced a DTA (Diphtheria toxin A)-Amp^{r} cassette as a negative selection marker 2 kb downstream of the LNL cassette with simultaneous removal of a 1 kb DNA fragment. FIG. 1A, Panel B shows that the NGAL-targeting construct was electroporated into the KV1 ES cells, and the neomycin-resistant ES clones were PCR-screened for the targeted NGAL allele from homologous recombination. ES cells that did not have homologous recombination were negatively selected by the presence of the DTA, while homologous recombination of the reporter construct in frame and downstream of the NGAL promoter were selected for. FIG. 1B, Panel C shows that ES cells with a Luc2-mCherry-LNL knockin allele were microinjected into wild-type C57B6 blastocysts to generate chimeric mice that were subsequently mated with albino C57B6 female mice for germline transmission. The F1 mice were PCR-genotyped by using primers F1, R1 and R2, and the germline transmitted mice contained both targeted (500 bp) and wild type alleles (300 bp). Verification of the germline-transmitted F1 mice. The targeted allele was PCR-amplified by using primers F2, and R3 which was from the 1 Kb DNA fragment replaced by a DTA-AMP^{r} cassette in the targeting construct. The mice with NGAL targeting through homologous recombination showed a band at 6819 bp. FIG. 1B, Panel D shows that NGAL-luciferase reponsiveness was validated in the NGAL reporter ES cells by exposure to nephrotoxin cyanide and TLR agonist, lipid A. Both stimulants induced NGAL-luciferase activity while the control conditions did not have any detectable NGAL reporter expression.

FIG. 2 shows in vivo bioluminescence imaging of the NGAL di-fusion reporter mouse was performed. Mice were challenged with 30 minutes of unilateral I/R and monitored for 24 hours using bioimager with both luciferase and mCherry reporter molecules as shown in Panel A. FIG. 2, Panel B shows photon intensity of the ischemic (closed red squares) and control contralateral kidney (closed blue diamonds) was plotted as a function of time for luciferase emission. The region of interest (ROI) was selected manually over both kidney regions and the area was kept constant overtime. Photon intensity in the y axis was recorded as the maximum intensity (photons (p) s⁻¹ cm⁻² sr⁻¹) within the ROI and displayed as a function of fold change of luciferase activity in the injured kidney versus the undamaged contralateral kidney. Panel C shows NGAL reporter kidney expression correlates to western blot of urinary NGAL. Panel D shows reporter activity is dose dependent and is reversible. Female reporter mice were challenged with either 15 (green line) or 30 (blue line) minutes of unilateral I/R and survived for 72 hours.

FIG. 3 shows NGAL reporter responds to lipidA stimulation. NGAL activity assayed by luciferase is upregulated and dose dependent in the kidney, liver, spleen, skin and lung 24 hours after lipidA stimulation at increasing doses (5, 15 and 30 mg/kg).

FIG. 4 shows that the damaged nephron tubule is the source of kidney NGAL. Panel A shows NGAL-mCherry visible in the medullary region of the ischemic kidney and absent from the control kidney using fluorescent microscopy of NGAL di-fusion reporter frozen sections. NGAL-mCherry expression is restricted to the nephron tubules revealed by anti-mCherry visualized by DAB chromogen (top image). The middle of Panel B shows NGAL-mCherry protein pattern reflects Ngal in situ hybridization pattern observed in wild type animals. The bottom image of Panel B reveals Ngal message is expressed in tubules during an lipidA challenge. FIG. 4, Panel C shows nephron is the source of uNGAL. Tubular localization of Ngal message in an ischemic kidney 24 hours after insult. Ngal message is found most heavily expressed in the Thick Ascending Limb of Henle in the outer-stripe of the outer medula, and the macula densa in the cortex, the collecting ducts of the inner medulla had moderate NGAL expression while the distal convoluted tubules of the cortex was observed at a lesser extent.

FIG. 5 shows that LipidA and cisplatin has a direct effect on renal epithelia. Plates seeded with cells from reporter kidneys were treated with LipidA (Panel A), and uropathogenic bacteria (CFT073) (Panel B) responded in dose-dependent manner. When these cells were pretreated (1 hour) with the Nf-κβ inhibitors, luciferase activity was reduced 15-80%.

FIG. 6 shows that volume depletion does not activate NGAL expression. Panel A shows NGAL reporter mouse does not upregulate NGAL-luciferase during a volume depleted state. Male mice tonically express NGAL in the testis, so we used male mice testis as an internal positive control. Panel B shows mice with volume depletion were hypernatremic with an average increase in serum sodium of 8 mmol/L (n=3, p<0.05). Panel C shows the rise in serum creatinine after 60 hours indicates a decrease in filtration typical of volume depletion.

FIG. 7 shows that cross transplants of NGAL knockout kidneys into wild type bodies and the reciprocal followed by ischemia reperfusion injury. Panel A shows Ngal message was present in the wild-type kidney in the wild-type to knockout transplant after FR while the Ngal message was absent in the liver in both conditions. These results show that NGAL message is derived from the kidney. Panel B shows urinary NGAL was present in the control (WT->WT) and the WT->KO transplant, while there was a significant decrease in uNGAL in the KO->WT transplant.

FIG. 8 shows that neutrophils do not contribute to urinary NGAL. Panel A shows FACS analysis of RB6-8C5 ablated mice show the neutrophil population absent indicated by region R1. Panel B shows Ngal message was still present in kidneys of neutrophil ablated mice as shown by in situ hybridization of NGAL.

### DETAILED DESCRIPTION OF THE INVENTION

### A. Definitions

Unless otherwise indicated, all terms used herein have the same meaning as they would to one skilled in the art of the present invention.

Luciferase, unless stated otherwise, includes prokaryotic and eukaryotic luciferases, as well as variants possessing varied or altered optical properties, such as luciferases that luminesce at wavelengths in the red range.

Light-generating is defined as capable of generating light through a chemical reaction or through the absorption of radiation.

Light is defined herein, unless stated otherwise, as electromagnetic radiation having a wavelength of between about 300 nm and about 1100 nm.

lux is defined as prokaryotic genes associated with luciferase and photon emission.

luc is defined as eukaryotic genes associated with luciferase and photon emission.

Heterologous gene refers to a gene which has been transfected into a host organism. Typically, a heterologous gene refers to a gene that is not originally derived from the transfected or transformed cells, genomic DNA.

Transgene refers to a heterologous gene which has been introduced, transiently or permanently, into the germ line or somatic cells of an organism.

### B. General Overview of the Invention

The present invention includes transgenic animals containing a heterologous gene construct encoding a light-generating protein or complex of proteins. The construct is driven by a selected promoter, and can include, for example, various accessory proteins required for the functional expression of the light-generating protein, as well as selection markers and enhancer elements.

Activation of the promoter results in increased expression of the genes encoding the light-generating molecules and accessory proteins. Activation of the promoter is achieved by the interaction of a selected biocompatible entity, or parts of the entity, with the promoter elements. If the activation occurs only in a part of the animal, only cells in that part will express the light-generating protein.

More specifically, the present invention related to a double fusion NGAL reporter animal compatible with bioluminescence and fluorescence imaging, to circumvent the complexity of serial blood and urine collection in a small animal, and to visualize NGAL expression in vivo. The di-fusion reporter construct contains at least two illuminating proteins, typically at least one fluorescent protein and at least one bioluminescent protein. Particularly effective proteins include a reporter gene encoding the bioluminescence Firefly luciferase (Luc2) reporter gene and a reporter gene encoding the monomeric red fluorescent protein (mCherry). Combination of these optical imaging strategies with the NGAL promoter gene in a single animal or a single cell line has the advantage of allowing low cost and high throughput screening of potential nephrotoxins, renal therapeutic or prophylactic agents, in real time. Each of the reporter elements provide the investigator a unique way of imaging NGAL expression in the animal.

These fluorescent elements complement one another so that NGAL can be visualized in the whole animal in vivo. The near red monomeric fluorescent molecule allows the animal to be visualized for protein expression in vivo and has the resolution for single cell resolution ex vivo. Luciferase complements mCherry because it is visible from deeper tissues.

The lipocalin NGAL is a bacteriostatic agent that is induced by bacterial, fungal infection, or by products of bacteria and fungi. It is also markedly stimulated by aseptic stimuli such as ischemia, hypoxia, medication toxicity, obstructive uropathy, cancers and neoplasias such as psoriasis.

Its measurement has been noted as a biomarker of AKI in many settings including ischemia reperfusion injury, sepsis, bacterial infection, drug toxicity, transplantation, obstructive uropathy, and in Chronic Kidney diseases which are progressive such as HIVAN.

The transgenic mouse and isolated cell lines of the present invention provides a tool to detect expression of NGAL in living animals and in specific cells derived from the mouse, including skin, liver, lung, kidney cells and their subtypes such as, in the kidney, proximal tubule cells, thin limb of Henle, thick limb of Henle, distal convoluted cells, collecting duct cells, macula densa cells, arterial, arteriolar, capillary, venous cells when they are exposed to the stressors listed above. This permits time course of onset, time course of decay, organ of origin and cell type of origin of NGAL expression. In addition, the reporter mouse can be mated with mice carrying genetic mutations that themselves cause kidney disease, allowing the mouse to detect and follow the onset of those diseases. Hence administration of infectious agents (bacteria, fungi, or their components), administration of toxins (cisplatin, gentamicin, non-steroidal anti-inflammatory drugs and others), administration of food contaminates (heavy metals such as lead, additives such as melamine), administration of ischemia or hypoxia, removal and reinplantation of the kidney, as in renal transplants, blockade of the kidney (by obstruction due to tumor, stone or vascular malformation), induce the expression of NGAL and hence activate the light emitting reporters from the NGAL-promoter reporter mice. In addition, it is also possible to mate the reporter mice with mice bearing genetic diseases (e.g. mice with mutations that cause polycystic kidney disease, cystic diseases of chronic kidney disease, cystic diseases of HIV kidney disease [Paragas et al, JASN 2009]) and to follow the onset of NGAL bioluminescence and fluorescence. Diseases of liver, heart, aorta and arteries, or diseases of the urogenital system that block urine flow including cancers of the ureter, bladder, prostate, uterus, or urinary stone diseases, or diseases of fibrosis, such as retroperitoneal fibrosis or uretral strictures, or dysmotility disorders of the bladder can each impact the kidney and induce AKI, are detectable by the reporter luminescence or fluorescence. A similar argument is made for all organs containing epithelia which can express NGAL, such as the skin (e.g. psoriasis, pemphigus, infection by bacteria or fungi), lung (e.g. cystic fibrosis, bronchitis, pneumonia), liver (alcoholic, hepatitic, medication, toxin, herbal induced cirrhosis) wherein the reporter mouse can detect the onset, severity, the resolution of the disease or treatment of the disease. In short, the ability to monitor NGAL gene expression in living cells in a mouse permits a broad range of pharmaceutical and toxicological and genetic research. Signal detection in real time in living animals means that the progression of a disease or a biological process can be studied throughout an experimental period without the traditional need to sacrifice mice at each data point. This results in higher quality data using fewer animals and ultimately speed the process of screening compounds leading to more rapid drug discovery, eliminates mouse to mouse variability, allows study of the effects of a second insult to the same mouse without confounders such as blood drawing, urine sampling, and finally the need to sacrifice the animal for pathological investigation.

The NGAL-Luc/mC construct measures signaling to the NGAL locus in vivo as a result of clinically significant stimuli. This signal has a variety of qualities relevant to the detection of clinical events including rapid expression (3-6 hours) and reversibility not only as a result of diminution of disease activity, but also as a result of pharmacological interventions which terminate the signal. In addition, organ specificity: ischemia to the kidney causes kidney specific expression, while LPS causes expression in a variety of organs. Lastly, it can be shown that kidney NGAL expression is Nfκβ-dependent in vivo.

The dependence on Nfκβ in vivo means that many clinically important inflammatory stimuli are detectable by Luc2/mC. But in addition, a second signal transduction pathway is detected based on hypoxia.

In sum, components of the kidney tubules, such as the proximal tubule, the TALH and CD of the kidney express NGAL as a secreted protein, which is released and lost into the urinary system. The invention provides a method to determine its site of expression in the cells that express NGAL by providing an accumulation of a light emitting protein in lieu of NGAL at the cellular sites of NGAL expression, as a rapid and robust response to a variety of agents found in our food, medications, environment or are the result of genetic diseases that impart damage to epithelial cells, reported by NGAL expression.

The contacting of a transgenic mammal with a candidate agent in accordance with the invention can include compositions that can include different types of pharmaceutically acceptable carriers, depending on whether they are to be administered in solid, liquid or aerosol form, and whether they need to be sterile for such routes of administration as injection. The candidate agent can include medications, toxins including chemicals and heavy metals, and poisons. The candidate agent and/or its composition can be in the form of a solid food, a liquid food, smoke, and fumes. The candidate agent and/or its composition can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, locally, or by inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art.

### C. Light-Generating Proteins

The light-generating moieties (LGMs), molecules or constructs useful in the practice of the present invention may take any of a variety of forms, depending on the application. They share the characteristic that they are luminescent, that is, that they emit electromagnetic radiation in ultraviolet (UV), visible and/or infra-red (IR) from atoms or molecules as a result of the transition of an electronically excited state to a lower energy state, usually the ground state.

Examples of light-generating moieties include photoluminescent molecules, such as fluorescent molecules, chemiluminescent compounds, phosphorescent compounds, and bioluminescent compounds.

Two characteristics of LGMs that bear considerable relevance to the present invention are their size and their spectral properties. Both are discussed in the context of specific types of light-generating moieties described below, following a general discussion of spectral properties.

An important aspect of the present invention is the selection of light-generating moieties that produce light capable of penetrating animal tissue such that it can be detected externally in a non-invasive manner. The ability of light to pass through a medium such as animal tissue (composed mostly of water) is determined primarily by the light's intensity and wavelength.

The more intense the light produced in a unit volume, the easier the light will be to detect. The intensity of light produced in a unit volume depends on the spectral characteristics of individual LGMs, discussed below, and on the concentration of those moieties in the unit volume. Accordingly, conjugation schemes that place a high concentration of LGMs in or on an entity (such as high-efficiency loading of a liposome or high-level expression of a bioluminescent protein in a cell) typically produce brighter light-emitting conjugates (LECs), which are easier to detect through deeper layers of tissue, than schemes which conjugate, for example, only a single LGM onto each entity.

A second factor governing the detectability of an LGM through a layer of tissue is the wavelength of the emitted light. Water may be used to approximate the absorption characteristics of animal tissue, since most tissues are composed primarily of water. It is well known that water transmits longer-wavelength light (in the red range) more readily than it does shorter wavelength light.

Accordingly, LGMs which emit light in the range of yellow to red (550-1100 nm) are typically preferable to LGMs which emit at shorter wavelengths. Several of the LGMs discussed below emit in this range. However, it will be noted, based on experiments performed in support of the present invention and presented below, that excellent results can be achieved in practicing the present invention with LGMs that emit in the range of 486 nm, despite the fact that this is not an optimal emission wavelength. It will be understood that through the use of LGMs with a more optimal emission wavelength, similar detection results can be obtained with LGEs having lower concentrations of the LGMs.

Fluorescence-based Moieties. Fluorescence is the luminescence of a substance from a single electronically excited state, which is of very short duration after removal of the source of radiation. The wavelength of the emitted fluorescence light is longer than that of the exciting illumination (Stokes' Law), because part of the exciting light is converted into heat by the fluorescent molecule.

Because fluorescent molecules require input of light in order to luminesce, their use in the present invention may be more complicated than the use of bioluminescent molecules. Precautions are typically taken to shield the excitatory light so as not to contaminate the fluorescence photon signal being detected from the subject. Obvious precautions include the placement of an excitation filter, such as that employed in fluorescence microscope, at the radiation source. An appropriately-selected excitation filter blocks the majority of photons having a wavelength similar to that of the photons emitted by the fluorescent moiety. Similarly a barrier filter is employed at the detector to screen out most of the photons having wavelengths other than that of the fluorescence photons. Filters such as those described above can be obtained from a variety of commercial sources, including Omega Optical, Inc. (Brattleboro, Vt.).

Alternatively, a laser producing high intensity light near the appropriate excitation wavelength, but not near the fluorescence emission wavelength, can be used to excite the fluorescent moieties. An x-y translation mechanism may be employed so that the laser can scan the subject, for example, as in a confocal microscope.

As an additional precaution, the radiation source can be placed behind the subject and shielded, such that the only radiation photons reaching the site of the detector are those that pass all the way through the subject. Furthermore, detectors may be selected that have a reduced sensitivity to wavelengths of light used to excite the fluorescent moiety.

Through judicious application of the precautions above, the detection of fluorescent LGMs according to methods of the present invention is possible.

Fluorescent moieties include small fluorescent molecules, such as fluorescein, as well as fluorescent proteins, such as green fluorescent protein (Chalfie, et al., 1994, Science 263:802-805., Morin and Hastings, 1971, J. Cell. Physiol. 77:313) and lumazine and yellow fluorescent proteins (O′Kane, et al., 1991, PNAS 88:1100-1104, Daubner, et al., 1987, PNAS 84:8912-8916). In addition, certain colored proteins such as ferredoxin IV (Grabau, et al., 1991, J Biol. Chem. 266:3294-3299), whose fluorescence characteristics have not been evaluated, may be fluorescent and thus applicable for use with the present invention. Ferredoxin IV is a particularly promising candidate, as it has a reddish color, indicating that it may fluoresce or reflect at a relatively long wavelength and produce light that is effective at penetrating tissue.

Commercially-available fluorescent molecules can be obtained with a variety of excitation and emission spectra that are suitable for use with the present invention. For example, Molecular Probes (Eugene, Oreg.) sells a number of fluorophores, including Lucifer Yellow (abs. at 428 nm, and emits at 535 nm) and Nile Red (abs. at 551 nm and emits at 636 nm). Further, the molecules can be obtained derivatized with a variety of groups for use with various conjugation schemes (e.g., from Molecular Probes).

Bioluminescence-Based Moieties. The subjects of chemiluminescence (luminescence as a result of a chemical reaction) and bioluminescence (visible luminescence from living organisms) have, in many aspects, been thoroughly studied (e.g., Campbell, 1988, Chemiluminescence. Principles and Applications in Biology and Medicine, Chichester, England: Ellis Horwood Ltd. and VCH Verlagsgesellschaft mbH). A brief summary of salient features follows.

Bioluminescent molecules are distinguished from fluorescent molecules in that they do not require the input of radiative energy to emit light. Rather, bioluminescent molecules utilize chemical energy, such as ATP, to produce light. An advantage of bioluminescent moieties, as opposed to fluorescent moieties, is that there is virtually no background in the signal. The only light detected is light that is produced by the exogenous bioluminescent moiety. In contrast, the light used to excite a fluorescent molecule often results in the fluorescence of substances other than the intended target. This is particularly true when the “background” is as complex as the internal environment of a living animal.

Several types of bioluminescent molecules are known. They include the luciferase family (e.g., Wood, et al., 1989, Science 244:700-702) and the aequorin family (e.g., Prasher, et al., Biochem. 26:1326-1332). Members of the luciferase family have been identified in a variety of prokaryotic and eukaryotic organisms. Luciferase and other enzymes involved in the prokaryotic luminescent (lux) systems, as well as the corresponding lux genes, have been isolated from marine bacteria in the Vibrio and Photobacterium genera and from terrestrial bacteria in the Xenorhabdus genus.

An exemplary eukaryotic organism containing a luciferase system (luc) is the North American firefly Photinus pyralis. Firefly luciferase has been extensively studied, and is widely used in ATP assays. cDNAs encoding luciferases from Pyrophorus plagiophthalamus, another species of click beetle, have been cloned and expressed (Wood, et al., 1989, Science 244:700-702). This beetle is unusual in that different members of the species emit bioluminescence of different colors. Four classes of clones, having 95-99% homology with each other, were isolated. They emit light at 546 nm (green), 560 nm (yellow-green), 578 nm (yellow) and 593 nm (orange). The last class (593 nm) may be particularly advantageous for use as a light-generating moiety with the present invention, because the emitted light has a wavelength that penetrates tissues more easily than shorter wavelength light.

Luciferases, as well as aequorin-like molecules, require a source of energy, such as ATP, NAD(P)H, and the like, and a substrate, such as luciferin or coelentrizine and oxygen.

The substrate luciferin must be supplied to the luciferase enzyme in order for it to luminesce. In those cases where a luciferase enzyme is introduced as an expression product of a vector containing cDNA encoding a lux luciferase, a convenient method for providing luciferin is to express not only the luciferase but also the biosynthetic enzymes for the synthesis of luciferin. In cells transformed with such a construct, oxygen is the only extrinsic requirement for bioluminescence. Such an approach, detailed in Example 1, is employed to generate lux-transformed Salmonella, which are used in experiments performed in support of the present invention and detailed herein.

The plasmid construct, encoding the lux operon obtained from the soil bacterium Xenorhabdus luminescens (Frackman, et al., 1990, J. Bact. 172:5767-5773), confers on transformed E. coli the ability to emit photons through the expression of the two subunits of the heterodimeric luciferase and three accessory proteins (Frackman, et al., 1990). Optimal bioluminescence for E. Coli expressing the lux genes of X. luminescens is observed at 37° C. (Szittner and Meighen, 1990, J. Biol. Chem. 265:16581-16587, Xi, et al., 1991, J. Bact. 173:1399-1405) in contrast to the low temperature optima of luciferases from eukaryotic and other prokaryotic luminescent organisms (Campbell, 1988, Chemiluminescence. Principles and Applications in Biology and Medicine, Chichester, England: Ellis Horwood Ltd. and VCH Verlagsgesellschaft mbH). The luciferase from X. luminescens, therefore, is well-suited for use as a marker for studies in animals.

Luciferase vector constructs, such as the one described above, can be adapted for use in transforming a variety of host cells, including most bacteria, and many eukaryotic cells (luc constructs). In addition, certain viruses, such as herpes virus and vaccinia virus, can be genetically-engineered to express luciferase. For example, Kovacs Sz. and Mettenlieter, 1991, J. Gen. Virol. 72:2999-3008, teach the stable expression of the gene encoding firefly luciferase in a herpes virus. Brasier and Ron, 1992, Meth. in Enzymol. 216:386-396, teach the use of luciferase gene constructs in mammalian cells. Luciferase expression from mammalian cells in culture has been studied using CCD imaging both macroscopically (Israel and Honigman, 1991, Gene 104:139-145) and microscopically (Hooper, et al., 1990, J. Biolum. and Chemilum. 5:123-130).

The lipocalin NGAL is a bacteriostatic agent, but it is also markedly stimulated by aseptic stimuli. Its measurement has been noted as a biomarker of AKI in many settings including ischemia reperfusion injury, sepsis, bacterial infection, drug toxicity, transplantation, obstructive uropathy, and in Chronic Kidney diseases which are progressive such as HIVAN.

NGAL protein has been identified as a renal biomarker for the detection and prognosis of acute and chronic renal tubular cell injuries, including acute and chronic renal failure, in both the urine and the serum portion of the blood, as described in US Patent Publications 2004-0219603, 2005-0272101, and 2007-0037232, the disclosures of which are incorporated herein by reference in their entirety. NGAL has also been identified as a therapeutic for the treatment, amelioration, reduction and prevention of acute and chronic organ injuries, including renal injuries, caused by ischemic injuries, ischemic-reperfusion injuries, and toxin-induced injuries, as described in US Patent Publication 2005-0261191, the disclosure of which is incorporated herein by reference in its entirety.

The transgenic mammal provides a tool to detect expression in living animals. This permits time course of onset, time course of decay, organ of origin. In short, the ability to monitor NGAL expression in living cells in a mouse permits a broad range of pharmaceutical and toxicological research. Signal detection in real time in living animals means that the progression of a disease or a biological process can be studied throughout an experimental period without the traditional need to sacrifice mice at each data point. This results in higher quality data using fewer animals and ultimately speeds the process of screening compounds leading to more rapid drug discovery

NGAL-Luc/mC measures signaling to the NGAL locus in vivo as a result of clinically significant stimuli. This signal has a variety of qualities relevant to the detection of clinical events including rapid expression (3-6 hours) and reversibility not only as a result of diminution of disease activity, but also as a result of pharmacological interventions which terminate the signal. In addition, organ specificity: ischemia to the kidney causes kidney specific expression, while LPS causes expression in a variety of organs. Lastly, kidney NGAL expression is Nfκβ-dependent in vivo. The dependence on Nfκβ in vivo means that many clinically important inflammatory stimuli are detectable by Luc2/mC. But in addition, a second signal transduction pathway is detected based on hypoxia.

In sum the TALH and CD of the kidney expresses a secreted protein into the urinary system as a rapid response, but in our new construct a light emitting reporter accumulates at the site of NGAL expression without being secreted and hence is a readily visible, quantitative, reversible, sensitive, specific indicator of NGAL expression and hence tissue stress.

### E. Methods

### 1. Gene Construction

Construction of pLuc-mCherry. Overlap PCR amplification and standard cloning techniques were used to fuse the mCherry gene from plasmid pmCherry (Clonetech PT3973-5) in frame with pLuc2 from plasmid and pGL4.10[luc2] (Promega, Madison, Wis.). For PCR amplifications, different 5′ and 3′ end primers were used to generate the fusion vectors and site-directed mutagenesis to ablate the stop codon on luc2. A 42 by spacer, 5′ cta gaa aac agc cat gcg agc gcg ggg tac cag get agc ace 3′, separate the reporter elements.

Other reporter constructs. The NGAL reporter mouse can be constructed with any of the available fluorescent and luciferase genes that represent 544-713 nm of the light spectrum and these genes are proven to express well in both mammalian cells and mice (see Table 1). All of these light-emitting molecules can be driven by the NGAL promoter for various applications in different organs and/or cell types allowing the visualization of cellular stress in vivo in real-time.

TABLE 1

| | Excitation maximum | Emission maximum |
|---|---|---|
| Fluorescent protein | (nm) | (nm) |
| infrared-fluorescent | 686 | 713 |
| proteins (IFPs) | | |
| mRFP1 | 584 | 607 |
| mCherry | 587 | 610 |
| mOrange | 548 | 562 |
| DsRed | 558 | 583 |
| tdTomato | 554 | 581 |
| mKO | 548 | 559 |
| TagRFP | 555 | 584 |
| EGFP or mEGFP | 488 | 507 |
| mOrange2 | 549 | 565 |
| mApple | 568 | 592 |
| TagRFP-T | 555 | 584 |
| Firefly Luciferase | | 550-570 |
| Renilla Luciferase | | 480 |
| Click Beetle Luciferase | | 544-593 |

Combinations of these light-emitting genes can be constructed to generate di- and tri-fusion constructs that will permit both in vivo bioluminescent imaging and fluorescent analysis of histological sections. For example, a fusion reporter with a luciferase and the infrared-fluorescent protein would permit the imaging of deep tissue with both fluorescence excitation and luminescence.

Validation of Luc2-mCherry di-fusion gene. 293T human embryonic kidney cells (American Type Culture Collection, Manassas, Va.) transfected with CMV-Luc2-mCherry were grown in MEM supplemented with 10% fetal bovine serum and 1% penicillin/streptomycin solution. All transient transfections are carried out using the Superfect transfection reagent (Qiagen). For quantification of the expression level of luc2 and mCherry present in the CMV-Luc2-mCherry, 1×10 4 293T cells expressing the vectors were seeded in clear 96-well plates and imaged in the Xenogen IVIS optical imaging system (Xenogen Corp., Almeda, Calif.) with a block excitation filter and an a block emission filter for fluorescence and a block emission filter for luciferase activity. For FACS, 1×10 6 of CMV-Luc2-mCherry infected 293T cells will be sorted.

Generation of a NGAL-di-fusion reporter BAC clone. Construction of a NGAL targeting construct by using a BAC recombineering strategy. The validated luciferase (Luc2)-mCherry di-fusion reporter gene together with a Loxp-Neomycin-Loxp cassette (LNL) was knocked into a site right before the translation initiation codon of the NGAL gene. A DTA (Diphtheria toxin A)-Amp^{r} cassette as a negative selection marker was then introduced to a site that is 2 kb downstream of the LNL cassette with simultaneous removal of a 1 kb DNA fragment. BAC recombineering was performed in a SW105 strain of E. coli by following a modified NCI protocol in the transgenic facility of Columbia University. Briefly, the mouse (C57) BAC clones with the NGAL gene were obtained from Children's Hospital Oakland Research Institute (CHORI), and transformed into SW105. The competent SW105 cells with BAC DNA and recombinatorial proteins were made and transformed with the DNA fragment above. The clones with recombination were selected on the basis of kanamycin resistance, and examined by PCR and DNA sequencing to assure the correct knockin.

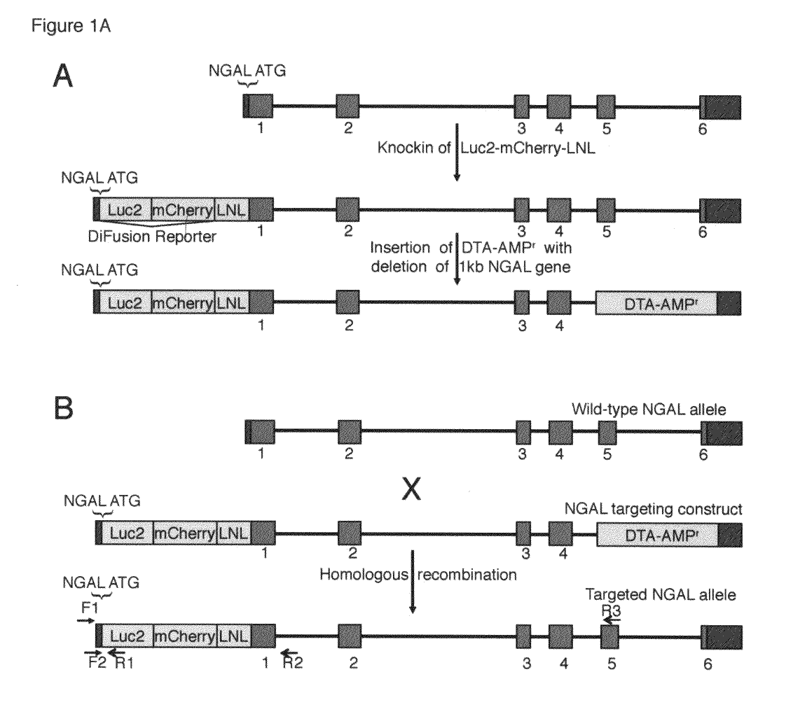

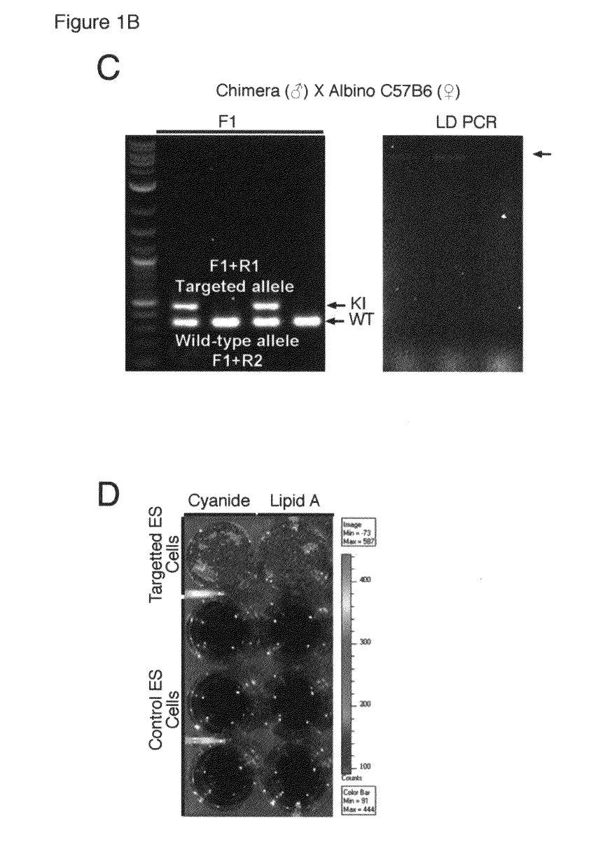

NGAL targeting in mouse ES cells. The NGAL-targeting construct was electroporated into the KV1 ES cells, and the neomycin-resistant ES clones were PCR-screened for the targeted NGAL allele for homologous recombination. Additionally, ES cells containing the NGAL di-fusion gene were challenged with either cisplatin or LPS to induce NGAL reporter activity (FIG. 1B, Panel D). ES cells with a Luc2-mCherry-LNL knockin allele were microinjected into wild-type C57B6 blastocysts to generate chimera which were subsequently mated with albino C57B6 female mice for germline transmission. The F1 mice were PCR-genotyped by using primers F1, R1 and R2, and the germline transmitted mice contained both targeted and wild type alleles (FIG. 1B, Panel C).

Verification of the germline-transmitted F1 mice. The targeted allele was PCR-amplified by using primers F2, and R3 which was from the 1 Kb DNA fragment replaced by a DTA-AMP^{r} cassette in the targeting construct. The mice with NGAL targeting through homologous recombination showed a band at 6819 bp (FIG. 1B, Panel C).

TABLE 2

| Nephron Segment: | Antigen: | Symbol: |
|---|---|---|
| Proximal Tubule (PT) | ¥ Aquaporin 1 | Aqp1 |
| | ¥ Chloride channel 5 | Clc5 |
| | ¥ Cubulin [Homo sapien]; lectin, galactose binding, soluble 3 | CUBN; Lgals3 |
| | [Mus musculus] | |
| | ¥ Leucine aminopeptidase | LAP |
| | ¥ Lotus Tetragonobulus lectin | LNP |
| | ¥ low density lipoprotein receptor-related protein 2 (Megalin) | Lrp2 |
| | ¥ solute carrier family 5 (sodium/glucose cotransporter), member 1 | Slc5al (Sglt1) |
| Thick Ascending Limb (TAL) | ¥ Chloride channel 5 | CLCN5 (Clck2) |
| | ¥ Uromodulin (Tamm-Horsfall protein) | Umod (THP) |
| | ¥ Solute carrier family 12, member 1 | Slc12a1 (Nkcc2) |
| | ¥ Polycystic kidney disease 2 | Pkd2 |
| | ¥ Potassium inwardly-rectifying channel, subfamily J, member 1 | Kcnj1 (Romk2) |
| | ¥ Epithelial membrane antigen | EMA |
| DT cells (TAL/distal | ¥ Epithelial membrane antigen | EMA |
| convoluted tubule (DCT)) | | |
| Distal Tubule (DT) | ¥ Chloride channel 5 | CLCN5 (Clck2) |
| | ¥ sodium channel, nonvoltage-gated, type I, alpha | Scnn1a (mENaC) |
| | ¥ solute carrier family 8 (sodium/calcium exchanger), member 1 | Slc8a1 |
| | ¥ Polycystic kidney disease 1 and 2 | Pkd1 and Pkd2 |
| | ¥ Potassium inwardly-rectifying channel, subfamily J, member 1 | Kcnj1 (Romk2) |
| | ¥ Epithelial membrane antigen | EMA |
| CD system cells (Connecting | | |
| Tubule (CT)/cortical CD) | | |
| Collecting Duct (CD) | ¥ L1 cell adhesion molecule | L1-CAM |
| Glomerulus | ¥ Actinin-4 | Actn4 |
| | ¥ CD2-associated protein | Cd2ap |
| | ¥ Cadherin 3 | Cdh3 |
| | ¥ Podocalyxin-like | Podx1 |
| | ¥ Podoplanin | Pdpn |

NGAL di-fusion primary cells from whole organs. Whole kidney cells were obtained from the NGAL di-fusion reporter mice (8-12 weeks of age) using collagenase (Sigma) digestion, differential sieving and seeded at a density of 1×10⁵ per well. Cells were washed with a buffered salt solution and grown overnight before exposure to TLR agonists, ROS inducing species or toxin. Plates were imaged in the Xenogen IVIS optical imaging system (Xenogen Corp., Almeda, Calif.) for NGAL-luciferase activity.

Pure populations of NGAL di-fusion primary cells. Fluorescence-activated cell sorting (FACS) is employed to isolate pure populations of cells from specific segments of the nephron out of the large mixed population of cells found throughout the kidney. Nephron segment specific antibodies will be used to tease apart these distinct renal compartments (Table 2). FACS sorted cells are grown on as described above. Other organs such as the heart, liver, lung, spleen, skin, and gut are also immuno-dissected to generate pure populations of NGAL di-fusion reporter cells. Primary cells are immortalized by viral-mediated induction of the large T-antigen, introduced through simian virus 40 (SV-40).

NGAL di-fusion reporter half-life. Reporter protein degradation (half-life) of the NGAL reporter constructs can be determined by induction of the NGAL reporter in cell culture in the presence of cycloheximide and examined by a luminometer for luminescent reporter activity or a fluorescence microscope for fluorescent reporter activity. After 24 hours, the induced cells can be treated with 100 mg/ml cycloheximide for 0, 1, 2, 3, 4, 5, and 6 hours to determine the how long the fusion protein will be stable inside of the cell. Luciferase half-life has been documented at being about 3 hours.

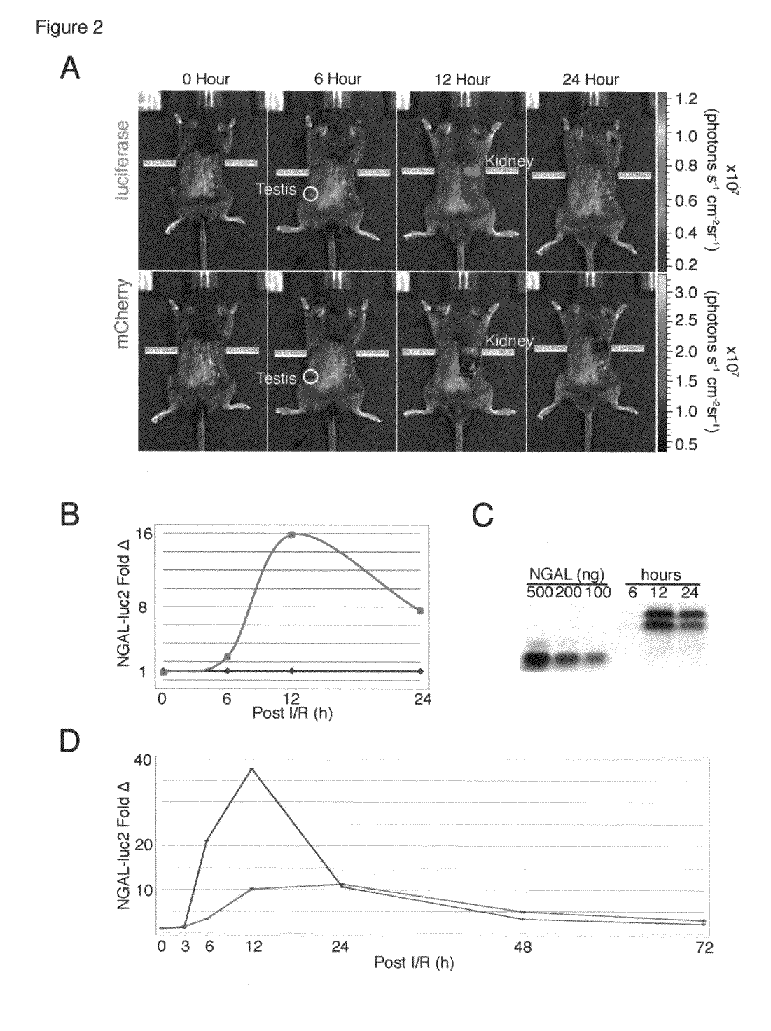

Breeding scheme. NGAL reporter mice are typically bred to generate a mouse that has one allele with the reporter genes occupying the promoted genomic site of the endogenous NGAL gene and the other allele having an undisturbed NGAL gene. With this model we show that NGAL secretion correlates to NGAL reporter activity (FIG. 2, Panel C). NGAL reporter animals can also be bred to generate a homozygous NGAL reporter with the reporter genes being on both alleles. This format effectively doubles the sensitivity of the screening system and permits the visualization of the slightest induction of the NGAL promoter. This model will be useful in drug-induced injury screening in a high-throughput cell culture system where only small amounts of novel pharmaceutical agents are available.

### 2. Stressors

NGAL induction. Techniques developed that model acute renal failure in the mouse are used to induce NGAL expression.
a. The kidney can be surgically challenged by ischemia reperfusion injury (I/R) and follow NGAL expression over time in vivo assayed by luminescence (FIG. 2, Panel A) and ex vivo by fluorescence microscopy. Animals will have serial blood drawn to compare the sensitivity of NGAL induction during renal failure with that of serum creatinine.
b. I/R injury can be performed and novel therapies assayed for efficacy in renal protection and limiting renal damage such as carbon monoxide (CO) donors, PARP inhibitor, fluids, ACE inhibitors, and other potential therapies to limit the expression of NGAL induction, limit renal failure, compared to the control.
Vera et al showed that administration of CO donor compounds tricarbonyldichlororuthenium(II) dimer, ([Ru(CO)3Cl2]2, 10 mg/kg) or tricarbonylchloro(glycinato)ruthenium(II) ([Ru(CO)3CI (glycinate)], (CORM-3) 1 hour before the onset of ischemia significantly decreased the levels of plasma creatinine 24 hours after reperfusion (Vera et al., 2005)
Conrad et al reported that the PJ34, a potent inhibitor of PARP, after the onset of acute hindlimb ischemia (post hoc) modulates the local production of inflammatory mediators during I/R (Conrad et al., 2006)
Himmelfarb et al suggests that early in the course of ARF, optimization of the hemodynamic status and correction of any volume deficit will have a beneficial effect on kidney function, will minimize further extension of the kidney injury, and will potentially facilitate recovery from ARF (Himmelfarb et al., 2008) further verifying NGAL's utility as a sensitive early predictor of kidney failure.
c. NGAL reporter mouse will be bred to various mouse models to elucidate NGAL's expression pattern in disease progression and investigate therapeutics to limit disease progression
NGAL reporter animal can be bred to the cpk mouse, a well-characterized recessive polycystic kidney disease (PKD) model, and various agents known to limit cyst growth will be applied such as taxol (Woo et al., 1994).
Strong induction of NGAL in the epidermis was seen in a variety of skin disorders characterized by dysregulated epithelial differentiation such as psoriasis, pityriasis rubra and squamous cell carcinoma. NGAL reporter animals will be bred with skin disorder animal models like the psoriasis model, a double knockout of JunB and c-Jun (Zenz et al., 2005) to permit testing of agents that reduce inflammation of the skin.
d. NGAL reporter mouse can be dosed with known nephrotoxic medications to determine the earliest point of nephrotoxicity and thus determine what point to suspend the medication prior to irreversible kidney injury.
e. NGAL reporter animals can receive cecal ligation and puncture (CLP), a surgical procedure that leads to sepsis. Kidneys responsiveness to full spectrum, full body sepsis is visualized in vivo.
f. NGAL reporter animals can be challenged by heavy metals such as lead which induce renal failure.
g. Bacteria and bacterial products such as Lipid A (Alexis Biochemicals 581-200-L002), Pam3Cys-SKKKK (EMC Microcollections, L2000), Escherichia coli Serotype 055:B5 Alexa Fluor 488 (495/519) (Molecular probes, L23351), Salmonella minnesota Alexa Fluor 488 (495/519) (Molecular probes, L23356), Pam3Cys-SKKKK(Aca-Aca-Fluorescein) (EMC Microcollections, L2034).
h. Cell stressors such as H₂O₂ (1 mmol/L for 4 hours), and Cobalt trigger NGAL expression.
i. Inhibitors, medication, and drugs can be tested to limit toxicity such as NfKB-inhibitor.

### 3. Imaging Techniques

Bioluminescence and Fluorescence Imaging of mCherry and luciferase Expression in Living Mice and primary cell lines. For in vivo bioluminescence imaging, mice were anesthetized, and injected with 150 mg/kg of luciferin in PBS (pH 7) via IP injection that is allowed to distribute in awake animals for about 10 minutes. The mice are anesthetized in a clear Plexiglas anesthesia box (2.5% isofluorane) that allows unimpeded visual monitoring of the animals. Mice are then placed in a light tight chamber equipped with a halogen light source, and whole body image was acquired for 30 seconds using the Xenogen IVIS optical imaging system with a block excitation filter and a block emission filter for NGAL-mCherry visualization and an open emission filter for NGAL-luciferase activity. Regions of interest (ROIs) were drawn on the dorsal side of the animal and quantified by using Living Image Software version 3.1. Counts detected in the ROIs by the CCD camera digitizer can by converted to physical units of radiance in photons/s/cm2/stcradian. Visualization of NGAL di-fusion reporter activity in primary cells were also assayed in the bioimager. NGAL reporter primary cells were first imaged for mCherry activity with block excitation and block emission and NGAL-luciferase activity was quantified by detection of light emitted after cell lysis and incubation with luciferin.

An important aspect of the present invention is the selection of a photodetector device with a high enough sensitivity to enable the imaging of faint light from within a mammal in a reasonable amount of time, preferably less than about 30 minutes, and to use the signal from such a device to construct an image.

In cases where it is possible to use light-generating moieties which are extremely bright, and/or to detect light-emitting conjugates localized near the surface of the subject or animal being imaged, a pair of “night-vision” goggles or a standard high-sensitivity video camera, such as a Silicon Intensified Tube (SIT) camera (e.g., Hamamatsu Photonic Systems, Bridgewater, N.J.), may be used. More typically, however, a more sensitive method of light detection is required.

In extremely low light levels, such as those encountered in the practice of the present invention, the photon flux per unit area becomes so low that the scene being imaged no longer appears continuous. Instead, it is represented by individual photons which are both temporally and spatially distinct from one another. Viewed on a monitor, such an image appears as scintillating points of light, each representing a single detected photon.

By accumulating these detected photons in a digital image processor over time, an image can be acquired and constructed. In contrast to conventional cameras where the signal at each image point is assigned an intensity value, in photon counting imaging the amplitude of the signal carries no significance. The objective is to simply detect the presence of a signal (photon) and to count the occurrence of the signal with respect to its position over time.

At least two types of photodetector devices, described below, can detect individual photons and generate a signal which can be analyzed by an image processor.

Reduced-Noise Photodetection Devices. The first class constitutes devices which achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately 120″ C. The “backthinned” refers to an ultra-thin backplate that reduces the path length that a photon follows to be detected, thereby increasing the quantum efficiency. A particularly sensitive backthinned cryogenic CCD camera is the “TECH 512”, a series 200 camera available from Photometrics, Ltd. (Tucson, Ariz.).

Photon Amplification Devices. A second class of sensitive photodetectors includes devices which amplify photons before they hit the detection screen. This class includes CCD cameras with intensifiers, such as microchannel intensifiers. A microchannel intensifier typically contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification.

An exemplary microchannel intensifier-based single-photon detection device is the C2400 series, available from Hamamatsu.

Image Processors. Signals generated by photodetector devices which count photons need to be processed by an image processor in order to construct an image which can be, for example, displayed on a monitor or printed on a video printer. Such image processors are typically sold as part of systems which include the sensitive photon-counting cameras described above, and accordingly, are available from the same sources (e.g., Photometrics, Ltd., and Hamamatsu). Image processors from other vendors can also be used, but more effort is generally required to achieve a functional system.

The image processors are usually connected to a personal computer, such as an IBM-compatible PC or an Apple Macintosh (Apple Computer, Cupertino, Calif.), which may or may not be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

Detection Field Of Device. The detection field of the device is defined as the area from which consistent measurements of photon emission can be obtained. In the case of a camera using an optical lens, the detection field is simply the field of view accorded to the camera by the lens. Similarly, if the photodetector device is a pair of “night vision” goggles, the detection field is the field of view of the goggles.

Alternatively, the detection field may be a surface defined by the ends of fiber-optic cables arranged in a tightly-packed array. The array is constructed to maximize the area covered by the ends of the cables, as opposed to void space between cables, and placed in close proximity to the subject. For instance, a clear material such as plexiglass can be placed adjacent the subject, and the array fastened adjacent the clear material, opposite from the subject.

The fiber-optic cable ends opposite the array can be connected directly to the detection or intensifying device, such as the input end of a microchannel intensifier, eliminating the need for a lens.

An advantage of this method is that scattering and/or loss of photons is reduced by eliminating a large part of the air space between the subject and the detector, and/or by eliminating the lens. Even a high-transmission lens, such as the 60 mm AF Nikkor macro lens used in experiments performed in support of the present invention, transmits only a fraction of the light reaching the front lens element.

With higher-intensity LGMs, photodiode arrays may be used to measure photon emission. A photodiode array can be incorporated into a relatively flexible sheet, enabling the practitioner to partially “wrap” the array around the subject. This approach also minimizes photon loss, and in addition, provides a means of obtaining three-dimensional images of the bioluminescence.

Other approaches may be used to generate three-dimensional images, including multiple detectors placed around the subject or a scanning detector or detectors.

It will be understood that the entire animal or subject need not necessarily be in the detection field of the photodetection device. For example, if one is measuring a light-emitting conjugate known to be localized in a particular region of the subject, only light from that region, and a sufficient surrounding “dark” zone, need be measured to obtain the desired information.

Immobilizing The Subject. In those cases where it is desired to generate a two-dimensional or three-dimensional image of the subject, the subject may be immobilized in the detection field of the photodetection devices during the period that photon emission is being measured. If the signal is sufficiently bright that an image can be constructed from photon emission measured in less than about 20 milliseconds, and the subject is not particularly agitated, no special immobilization precautions may be required, except to insure that the subject is in the field of the detection device at the start of the measuring period.

If, on the other hand, the photon emission measurement takes longer than about 20 msec, and the subject is agitated, precautions to insure immobilization of the subject during photon emission measurement, commensurate with the degree of agitation of the subject, need to be considered to preserve the spatial information in the constructed image. For example, in a case where the subject is a person and photon emission measurement time is on the order of a few seconds, the subject may simply be asked to remain as still as possible during photon emission measurement (imaging). On the other hand, if the subject is an animal, such as a mouse, the subject can be immobilized using, for example, an anesthetic or a mechanical restraining device.

A variety of restraining devices may be constructed. For example, a restraining device effective to immobilize a mouse for tens of seconds to minutes may be built by fastening a plexiglass sheet over a foam cushion. The cushion has an indentation for the animal's head at one end. The animal is placed under the plexiglass such that its head is over the indentation, allowing it to breathe freely, yet the movement of its body is constrained by the foam cushion.

In cases where it is desired to measure only the total amount of light emanating from a subject or animal, the subject does not necessarily need to be immobilized, even for long periods of photon emission measurements. All that is required is that the subject be confined to the detection field of the photodetector during imaging. It will be appreciated, however, that immobilizing the subject during such measuring may improve the consistency of results obtained, because the thickness of tissue through which detected photons pass will be more uniform from animal to animal.

Fluorescent Light-Generating Moieties. The visualization of fluorescent light-generating moieties requires an excitation light source, as well as a photodetector. Furthermore, it will be understood that the excitation light source is turned on during the measuring of photon emission from the light-generating moiety.

Appropriate selection of a fluorophore, placement of the light source and selection and placement of filters, all of which facilitate the construction of an informative image, are discussed above, in the section on fluorescent light-generating moieties.

High-Resolution Imaging. Photon scattering by tissue limits the resolution that can be obtained by imaging LGMs through a measurement of total photon emission. It will be understood that the present invention also includes embodiments in which the light-generation of LGMs is synchronized to an external source which can be focused at selected points within the subject, but which does not scatter significantly in tissue, allowing the construction of higher-resolution images. For example, a focused ultrasound signal can be used to scan, in three dimensions, the subject being imaged. Light-generation from areas which are in the focal point of the ultrasound can be resolved from other photon emission by a characteristic oscillation imparted to the light by the ultrasound (e.g., Houston and Moerner, U.S. Pat. No. 4,614,116, issued Sep. 30, 1986.)

Constructing An Image Of Photon Emission. In cases where, due to an exceptionally bright light-generating moiety and/or localization of light-emitting conjugates near the surface of the subject, a pair of “night-vision” goggles or a high sensitivity video camera was used to obtain an image, the image is simply viewed or displayed on a video monitor. If desired, the signal from a video camera can be diverted through an image processor, which can store individual video frames in memory for analysis or printing, and/or can digitize the images for analysis and printing on a computer.

Alternatively, if a photon counting approach is used, the measurement of photon emission generates an array of numbers, representing the number of photons detected at each pixel location, in the image processor. These numbers are used to generate an image, typically by normalizing the photon counts (either to a fixed, pre-selected value, or to the maximum number detected in any pixel) and converting the normalized number to a brightness (greyscale) or to a color (pseudocolor) that is displayed on a monitor. In a pseudocolor representation, typical color assignments are as follows. Pixels with zero photon counts are assigned black, low counts are assigned blue, and increasing counts are assigned colors of increasing wavelength, on up to red for the highest photon count values. The location of colors on the monitor represents the distribution of photon emission and, accordingly, the location of light-emitting conjugates.

In order to provide a frame of reference for the conjugates, a greyscale image of the (still immobilized) subject from which photon emission was measured is typically constructed. Such an image may be constructed, for example, by opening a door to the imaging chamber, or box, in dim room light, and measuring reflected photons (typically for a fraction of the time it takes to measure photon emission). The greyscale image may be constructed either before measuring photon emission, or after.

The image of photon emission is typically superimposed on the greyscale image to produce a composite image of photon emission in relation to the subject.

If it desired to follow the localization and/or the signal from a light-emitting conjugate over time, for example, to record the effects of a treatment on the distribution and/or localization of a selected biocompatible moiety, the measurement of photon emission, or imaging can be repeated at selected time intervals to construct a series of images. The intervals can be as short as minutes, or as long as days or weeks.

Analysis Of Photon Emission Images. Images generated by methods and/or using compositions of the present invention may be analyzed by a variety of methods. They range from a simple visual examination, mental evaluation and/or printing of a hardcopy, to sophisticated digital image analysis. Interpretation of the information obtained from an analysis depends on the phenomenon under observation and the entity being used.

### 4. NGAL Quantification Testing

Western Blot. NGAL was quantified by western blots, using non-reducing 4-15% tris-HCL gels (Bio-Rad, Laboratories, Inc. Hercules, Calif.) and monoclonal (1:1000; AntibodyShop, Gentofte, Denmark) or rabbit polyclonal antibodies (R&D Systems, Minneapolis) together with standards (0.2-10 ng) of human or mouse recombinant NGAL protein. NGAL was reproducibly detected to 0.4 ng/lane. NGAL expression was quantified using ImageJ software (NIMH).

In situ hybridization. NGAL RNA was detected using digoxigenin-labeled antisense riboprobes generated from cDNAs encoding Ngal (exon 1-7, 566 bp) by linearization with XhoI followed by T7 RNA polymerase. Kidneys were collected in ice-cold PBS and fixed overnight at 4° C. in 4% paraformaldehyde (PFA) in 0.1 M phosphate buffer saline (PBS), briefly quenched in 50 mM NH₄Cl, cryoprotected overnight in 30% sucrose PBS and embedded and sectioned (16 μM) in O.C.T. compound. The sections were post-fixed in 4% PFA for 10 min, treated with proteinase K (1 mg/ml for 3 min), acetylated and prehybridized for 2 hrs, and then hybridized at 68-72° C. overnight. The prehybridization and hybridization solution was 50% formamide, 5′ SSC, 5′ Denhardts, 250 mg/ml baker's yeast RNA (Sigma), and 500 mg/ml herring sperm DNA (Sigma). Sections were washed at 72° C. in 5′ SSC for 5-10 minutes, then at 72° C. in 0.2° SSC for 1 hour and then stained overnight (4° C.) with an anti-digoxigenin antibody coupled with alkaline phosphatase (Boehringer-Mannheim), at a 1:5000 dilution in 0.1 M Tris-HCl, pH 7.5, 0.15 M NaCl, 1% heat inactivated goat serum. Alkaline phosphatase activity was detected using BCIP, NBT (Boehringer-Mannheim) with 0.25 mg/ml levamisole in a humidified chamber for 1-3 days in the dark. Sections were dehydrated and mounted in Permount (Fisher Scientific).

Total RNA was isolated with the mirVANA RNA extraction kit (Ambion).

Real-Time PCR were prepared according to Bio-Rad SYBR GREEN, iCyclerMyiQ protocols. Target genes, including Ngal, β-actin, utilized respectively: Ngal 116 forward primer 5′-ctcagaacttgatccctgcc-3′ and NGALa593 reverse 5′-tccttgaggcccagacactt-3′; β-actin415 forward primer 5′-ctaaggccaaccgtgaaaag-3′ and β-actin 696 reverse primer 5 ‘-tctcagctgtggtggtgaag-3 ’. The ΔΔCT method was used to calculate fold amplification of transcripts.

Real Time PCR analysis. Samples were processed according to Bio-Rad SYBR GREEN, iCyclerMyiQ protocols. Target genes utilized respectively: Ngal 116 forward primer 5′-ctcagaacttgatccctgcc-3′ and NGALa593 reverse 5′-tccttgaggcccagacactt-3′; β-actin415 forward primer 5′-ctaaggccaaccgtgaaaag-3′ and β-actin 696 reverse primer 5′-tctcagctgtggtggtgaag-3′. The ΔΔCT method was used to calculate fold amplification of transcripts.

RNA isolation. Microarrays and real time PCR utilized RNA isolated with the mirVANA RNA extraction kit (Ambion) and quantified by NanoDrop and gel electrophoresis.

Kidney transplantation. For kidney transplantation, the donor's abdominal cavity is opened by a large longitudinal incision. Abdominal contents are reflected to the left side of the animal exposing the IVC. The IVC is cannulated and as much blood as possible is aspirated. 1 mL of cold heparinized saline (10 units/mL) is administered via the IVC using another 1-ml syringe. The kidneys are removed from the retroperitoneum with aorta and vena cava en bloc. The ureters are removed with a large patch of bladder. A midline laparotomy incision will be made using sterile surgical instruments using a 10× dissecting microscope. The small intestine is gently reflected superiorly. The intestines are covered with gauze and kept moist throughout the procedure with sterile saline. The recipient's left kidney is removed following ligation of the vessels and cauterization of the ureter. The recipient's abdominal IVC and aorta are exposed from the renal vessels to the iliac bifurcation. The donor aorta and IVC are anastomosed to the recipient's aorta and IVC using 10-0 suture. The kidney graft is reperfused. Two holes are made in the recipient bladder. The distal 5 mm of donor ureter is pulled into the recipient's bladder. The donor ureter is fixed to the exterior wall of the recipient bladder using 10-0 suture. The intestines are returned to the abdominal cavity. The abdominal incision will be closed in 2 layers. The muscle and fascia will be closed using interrupted 5-0 maxxon stitch. The skin will closed in running 5-0 biosyn. Four days after transplantation, the previous incision will be opened and the recipient right kidney is removed. The vascular pedicle of the kidney will be ligated with 7-0 silk. After the second surgery, the abdominal incision will be closed in 2 layers. The muscle and fascia will be closed using interrupted stitch 5-0 Maxxon. The skin will be closed in running 5-0 biosyn. Both closure sutures are absorbable so that suture removal is not necessary. Each animal will be given a sub-cutaneous injection of Lactated Ringers (1-2 cc), warmed to body temperature, and then be placed on warming water blanket. Supplemental oxygen will be administered during and immediately post-op to minimize hypoxia. There will be napanectar and food on the animals' cage floor in a petri dish for the first 24 hours post-op. Each animal is monitored for two weeks or until serum creatinine has stabilized to 0.5 mg/dL and there is no uNGAL.

### F. Examples

### Example #1

### Construction of NGAL Di-Fusion Mouse

Luciferase (pGL4.10[luc2], Promega E6651) and mCherry (pmCherry [mC], Clontech 632522) genes separated by a 42 by spacer were ligated by overlap PCR and validated in 293T cells using a CMV promoter to drive robust Luc2 and mC bioluminescence and fluorescence (FIG. 1A, Panel A). The Luc2-mCherry di-fusion construct was then inserted in frame at the endogenous mouse NGAL start site (nucleotide 32240385, chromosome 2) by bacterial recombineering (FIG. 1A, Panel B). The excised NGAL-luc2-mCherry insert (FIG. 1A, Panel B) was electroporated into EL250/RP23-192A7 ES cells and homologous recombination identified using genomic forward primerF1 and either a luciferase specific reverse primerR1 or a genomic reverse primerR2 (FIG. 1B, Panel C). The integration and orientation of the luc2-mC fusion reporter was verified by LD-PCR (FIG. 1B, Panel C) and sequencing (data not shown), and its functional state was demonstrated by treating knockin ES clones with lipidA and cisplatin, a stimulus that typically activates NGAL expression in vivo (FIG. 1B, Panel D).

Di-fusion reporter mouse responds to Ischemia Reperfusion and LipidA.

We challenged the NGAL/Luc2mC mouse with ischemia reperfusion injury (I/R) of the kidney, and measured reporter bioluminescence and fluorescence in vivo. After 30 minutes of unilateral I/R, NGAL/luc2-mC activity was specifically located in the operated kidney; neither the untouched contralateral kidney nor other organs expressed either reporter gene (FIG. 2, Panel A). The intensity of reporter expression depended on the ischemic dose; for example, NGAL-Luc2 activity was 40 fold higher after a 30 min dose of ischemia, but only 10 fold higher after 15 min of ischemia compared to contralateral kidney (FIG. 2, Panel B). Reporter expression was also rapid; bioluminescence was detected 3-6 hours after the insult. Expression also demonstrated a large dynamic range; 1-2 log fold changes were typically observed after 30 min of ischemia (FIG. 2, Panel B).

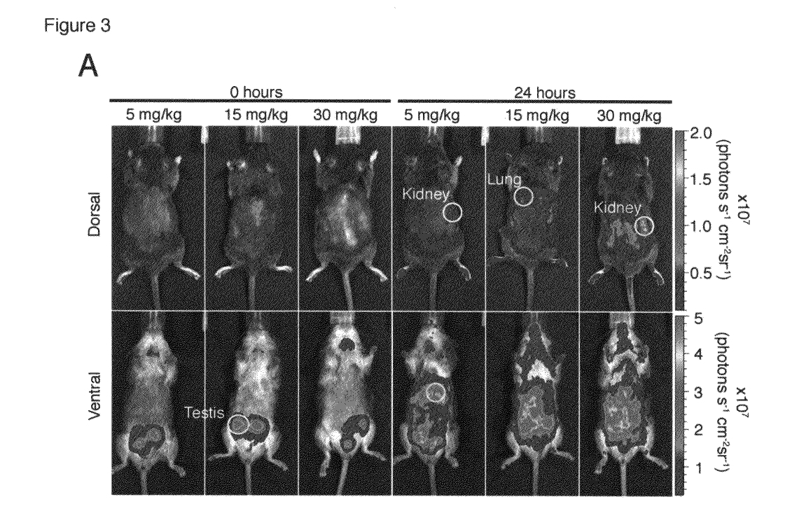

We found that LipidA, the purified lipid component of an endotoxin lipopolysaccharide, was a second stimulus for Luc2/mC expression. Titrating the dose of LipidA (i.p.) resulted in a graded Luc-mC response by a variety of organs in the living animal (FIG. 3, dorsal and ventral). These data demonstrate dose-responsive Luc2/mC activity in response to LipidA. Luc2/mC activity paralleled the NGAL message induced by LipidA (30 mg/kg for example induced 30 fold in the kidney) or induced by cecal-ligation and puncture (50 fold increase in signal, data not shown), demonstrating that Luc2/mC faithfully reports NGAL expression in response to a bacterial stimulus.

Hence, NGAL-Luc2/mC mouse provides a method for detection of stimuli that damage kidney epithelia in vivo. The locus of Luc2/mC expression depends on the site of the stimulus, and the intensity of Luc2/mC expression depends on the dosage of the stimulus.

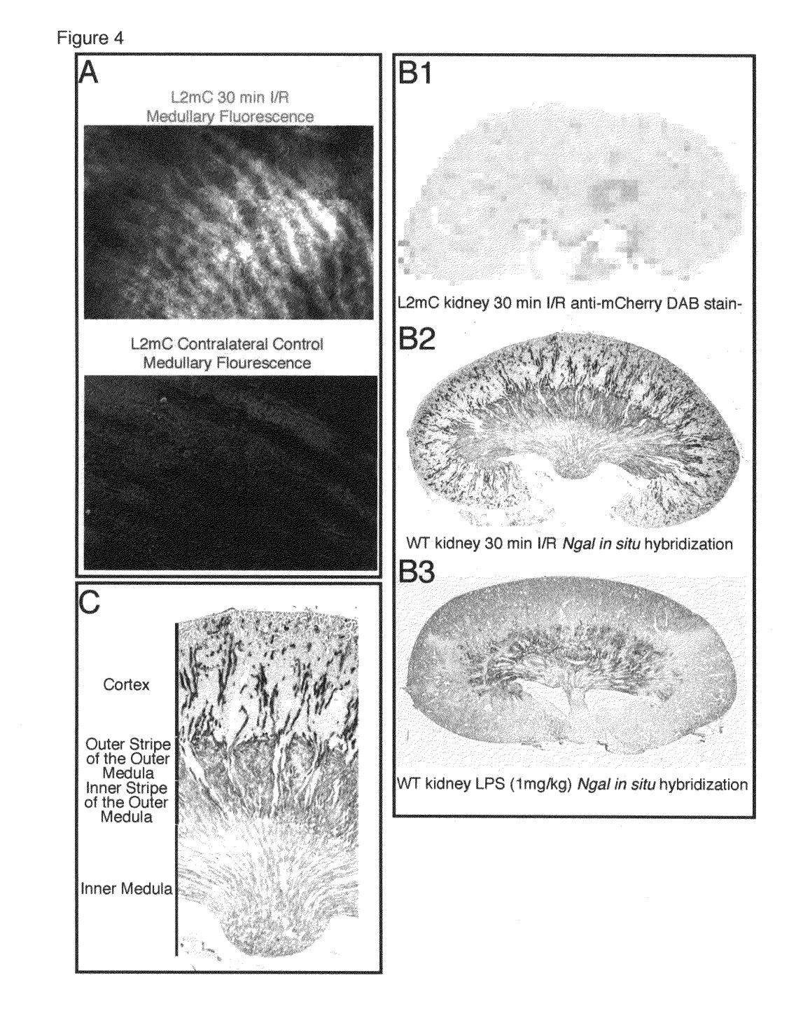

The damaged nephron tubule is the source of kidney NGAL. The NGAL reporter responded to both septic and aseptic stimuli; here, we show that the nephron tubule is the origin of NGAL Luc/mC bioluminescence and fluorescence after both types of stimuli. First, we sectioned the reporter kidneys that we had subjected to I/R, and by fluorescence microscopy found that tubules located in the medullary region of the kidney where the source of mC (FIG. 4, Panel A). Next, for finer localization of the reporters, we performed mC immunohistochemistry, and found that mC was expressed by tubules, in a pattern typical of Thick Limb of Henle and Collecting Ducts (FIG. 4, Panel B). The Luc2/mC pattern was essentially identical to sites of expression of endogenous NGAL message in the TALH, including the macula densa (MD) and in CDs, with weaker expression in the distal convoluted tubule (DCT), and no expression in proximal tubules (FIG. 4, Panel B and Panel C). A similar pattern was found after LipidA treatment, but with predominate expression in inner medullary tubules (FIG. 4, Panel B). These data indicate that Luc2/mC is expressed by specific segments of the nephron, reproducing the pattern of NGAL gene expression.

To determine whether Luc2/mC expression originates from tubules containing evidence of cellular disruption rather than simply from ‘bystander’ nephrons, we compared the distribution of mC (immunodetection) with the distribution of intra-luminal casts (H&E and PAS) reflecting cell shedding in damaged tubules. This demonstrated that mC was expressed by damaged tubules.

In sum, NGAL Luc2-mC and NGAL message colocalized in tubular segments associated with nephron injury as visualized by the presence of damaged cells in the lumen of the tubules.

Identification of Hidden Damage. Serum creatinine was little changed in our model of unilateral ischemia, because the untouched contralateral kidney limited the opportunity for azotemia. Hence, NGAL Luc/mC detected unilateral kidney disease, whereas serum creatinine was insensitive to unilateral disease. Moreover, while LipidA injection induced Luc/mC and azotemia at a 30 mg/kg dose of LipidA, low doses of LipidA also induced Luc/mC but without azotemia or a change in serum creatinine, highlighting the sensitivity of the reporter construct.

### Example #2

### Reversible Expression of NGAL Luc-mC

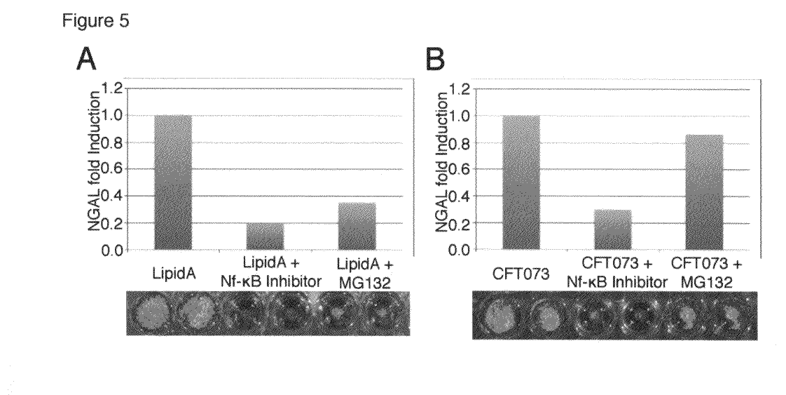

A signaling pathway known to activate NGAL expression was taken advantage of in order to examine whether Luc2/mC expression was reversible after cessation of the stimulus. Ligation of bacterial components by Toll-like receptors (TLR) has been shown to stimulate NGAL expression (Flo et al), for example, activation of TLR4 by LipidA. Subsequently, Nf-κβ mediates NGAL transcription. Hence, to determine whether pharmacological intervention could terminate NGAL expression in vivo, we utilized two inhibitors of Nf-κβ signaling, MG-132, a selective proteasome inhibitor, which inhibits NFkappaB activation by preventing IkappaB degradation and CU-160, a novel inhibitor of Nf-κβ signaling (Gong, Bioorganic and Medicinal chemistry letters 2009). We found that both these agents reduced LipidA induced Luc/mC expression (FIG. 5). In addition, the testis is one of the few sites that tonically expresses NGAL and Luc2/mC, and even this site was suppressed by these inhibitors.

To determine whether the inhibition by MG-132 and CU-160 constituted a direct effect on renal epithelia, we seeded plates with cells from reporter kidneys and treated them with LipidA. We found that the primary cells responded in dose-dependent manner to LipidA (FIG. 5). When these cells were pretreated (1 hour) with the Nf-κβ inhibitors, luciferase activity was reduced 15-100% (FIG. 5).

To test the reversibility of NGAL Luc/mC using clinically relevant interventions, we infected primary kidney cell cultures with uropathogenic E. coli. Reporter expression was noted over 1-3 days after an initial innoculum of bacteria. In fact, colony counts at the end of the culture correlated with Luc expression. On the other hand, when antibiotics were added, as a pretreatment (1 hr) or a posttreatment (1-12 hours), the signal was reversed. In a second clinically relevant model, we found that hypoxia induced Luc2/mC activation, but returning the primary cells to reoxygenated conditions suppressed the Luc2/mC signal to baseline.

### Example #3

### Volume Depletion does not Activate NGAL Expression

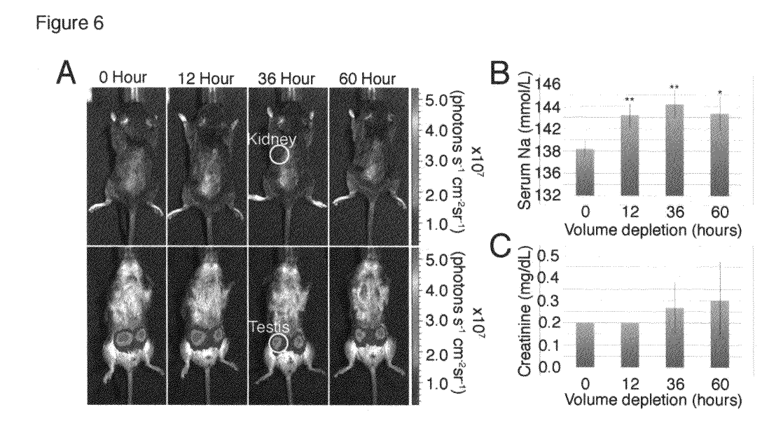

LipidA and ischemia-reperfusion injury, both induce “acute kidney injury”, which if extensive results in a graded rise in serum creatinine and a fall in glomerular filtration rate. However, volume depletion (pre-renal azotemia) also elevates serum creatinine and reduces the glomerular filtration rate. However, the later is a physiological adaptation characterized by few pathological changes in the urinary system and rapid reversibility (hours), while the former results in distinctive pathological changes in nephron epithelia, a prolonged reduction in GFR (˜days), and well known increases in morbidity and mortality. We examined these fundamental distinct aspects of renal function in NGAL-Luc2/mC mice and found that volume depletion is sufficient to produce hypernatremia (140.3 mmol/L to 148.3 mmol/L), reduced body weight by 25%, and a doubling of the serum creatinine (FIG. 6, Panel A and B) failed to induce NGAL-Luc2/mC expression (FIG. 6, Panel A and C). In contrast, as a positive control, the addition of LipidA effectively induced Luc/mC in the same mice at a later time. These data demonstrated that kidney expression of NGAL-Luc/mC distinguished volume depletion from acute kidney injury despite decreased GFR in both settings. Our finding parallels studies in human cohorts, whereby uNGAL can distinguish a patient with reversible pre-renal azotemia from acute kidney injury with a poorer prognosis.

The kidney is the source of urinary NGAL (uNGAL). In human studies, NGAL located in the urine pool (uNGAL) or in the serum pool (sNGAL) have been used as surrogates for kidney expressed NGAL. However, the relationship between kidney NGAL and either surrogate pool has not been proven.

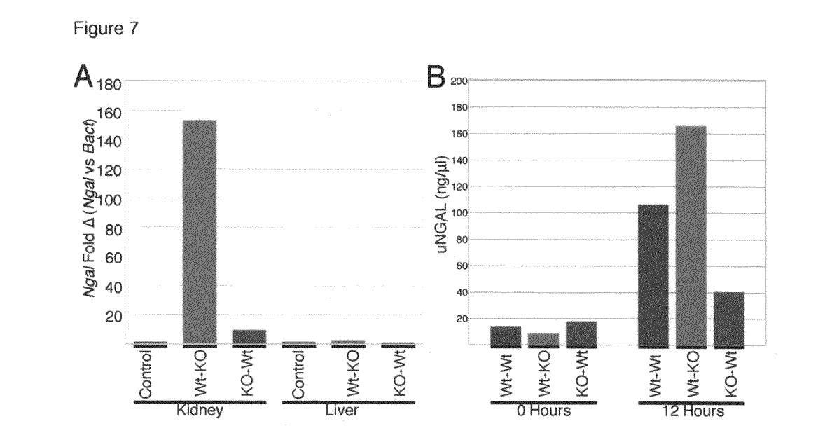

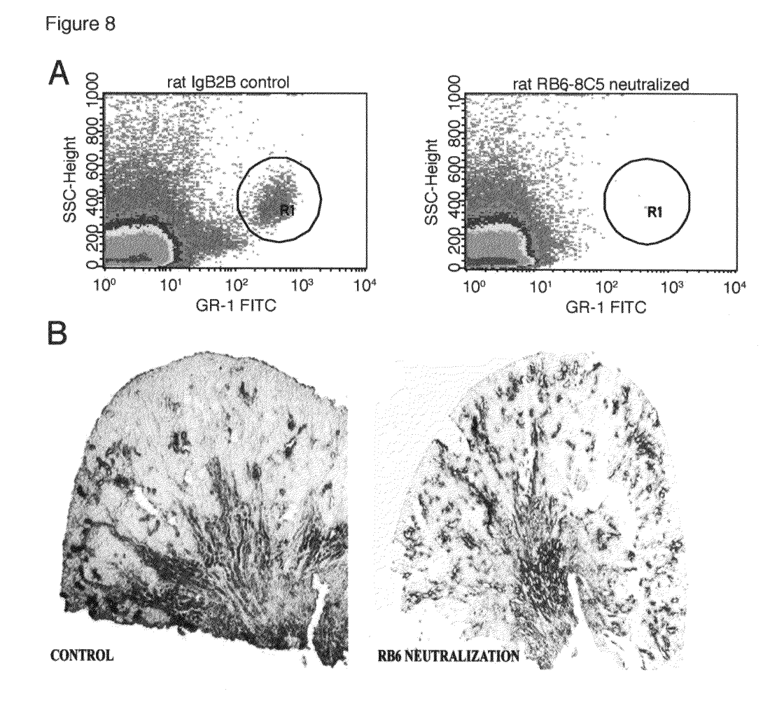

Here we studied the urinary pool. According to data from the reporter mouse, kidney Luc2/mC uNGAL is expressed simultaneously with uNGAL. To prove this association, we placed NGAL^{-/-} kidneys into NGAL^{+/+} bodies or conversely NGAL^{+/+} kidneys into NGAL^{-/-} bodies. Two weeks after graft maturation, we first confirmed that uNGAL and serum creatinine were identical to normal values, and then we administered a low dose of ischemia (10 minutes) directly to the transplanted kidney. FIG. 7, Panel A depicts a 150 fold increase in NGAL RNA (real time PCR) from NGAL^{+/+} kidneys in a NGAL^{-/-} body, whereas NGAL^{-/-} kidney in a NGAL^{+/+} body had 1/30 the concentration of kidney message (FIG. 7, Panel A), perhaps reflecting some infiltrating NGAL^{+/+} blood derived cells post-transplantation. Further, in this model, the liver failed to express NGAL at levels greater than control, reflecting the specificity of surgical manipulation. Consistently, uNGAL protein arose from the NGAL^{+/+} kidney graft placed in the NGAL^{+/+} or in the NGAL^{+/+} body, whereas cross transplanted animals containing NGAL^{-/-} kidneys in NGAL^{+/+} bodies showed little increase in the amount of uNGAL (FIG. 7, Panel B). These data revealed that the post-ischemic kidney appears to be the principal source of uNGAL (as well as contributing to the pool of serum NGAL, data not shown), suggesting that Luc2/mC has the diagnostic power of uNGAL. Further, it should be noted that while neutrophils may contribute to uNGAL by invading the ischemic organ, the deletion of neutrophils by the RB-6 neutralizing antibody (FIG. 8) failed to limit the expression of NGAL message or uNGAL protein after ischemic treatment of the kidneys, findings which were consistent with the notion that uNGAL derives from kidney epithelia, rather than from other cell types.

What is claimed is:

1. A transgenic mammal, including a transgenic mouse, whose genome comprises a transgene, said transgene comprises a neutrophil gelatinase-associated lipocalin (NGAL) promoter gene operably linked to at least one sequence encoding at least one of a fluorescent or bioluminescent protein, wherein the NGAL promoter gene expression in the mouse can be assayed by bioluminescence or fluorescence imaging.

2. The transgenic mammal of claim 1, wherein the protein includes a luciferase protein and a red fluorescent protein, and the sequence encoding the luciferase protein and the sequence encoding the red fluorescent protein are separated by a spacer.

3. The transgenic mammal of claim 1, wherein the luciferase protein is firefly luciferase (Luc2) protein.

4. The transgenic mammal fluorescent protein of claim 1, wherein the red fluorescent protein is monomeric red fluorescent protein (mCherry).

5. A progeny of the transgenic mammal of claim 1, wherein the genomes of said progeny comprise said transgene, and NGAL gene expression in the progeny can be assayed by bioluminescence or fluorescence imaging.

6. A method of screening for candidate agent that would cause organ injury including renal injury, comprising the steps of:
(a) contacting the transgenic mammal of claim 1 with an agent; and
(b) examining NGAL expression in the transgenic mouse by bioluminescence or fluorescence imaging, wherein increased bioluminescence or fluorescence after treatment with the agent indicates the agent would cause organ or tissue injury.

7. A method of screening for candidate agent that would prevent or treat injury to an organ or tissue that is detected by expression of NGAL, comprising the steps of:
(a) inducing renal injury in the transgenic mammal of claim 1;
(b) contacting the transgenic mouse with an agent; and
(c) examining NGAL expression in the transgenic mouse by bioluminescence or fluorescence imaging, wherein decreased bioluminescence or fluorescence after treatment with the agent indicates the agent would prevent or treat injury.

8. The method of claim 7, wherein the injury is renal injury, renal failure or sepsis.

9. A transgenic cell line comprising an neutrophil gelatinase-associated lipocalin (NGAL) promoter gene operably linked to at least one sequence encoding at least one of a fluorescent or bioluminescent protein, wherein the NGAL promoter gene expression in the cell line can be assayed by bioluminescence or fluorescence imaging.
